(19) United States
(12) Patent Application Publication
(10) Pub. No.: US 2006/0035301 A1
(43) Pub. Date: Feb. 16, 2006

(54) Method of identifying protein kinase modulators and uses therefore

(75) Inventors: Silvia Corvera, Westborough, MA (US); Leanne Wilson-Fritch, Auburn, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 11/044,652

(22) Filed: Jan. 26, 2005

Related U.S. Application Data
(60) Provisional application No. 60/539,420, filed on Jan. 26, 2004.

(51) Int. Cl. C12Q 1/48

(52) U.S. Cl. 435/15

(57) ABSTRACT

Methods of identifying AMPK or AK modulators are provided. In particular, methods that feature identifying modulators of AK and its associated substrates (including AMP, ATP or AMP and ATP) or AMPK or activities associated therewith, are provided. Therapeutic methods utilizing compounds identified according to the methods of the invention are also provided.

### RELATED APPLICATIONS

This application claims the benefit of U.S. Ser. No. 60/539,420, entitiled “Method of Identifying Protein Kinase Modulators and Uses Therefore” filed on Jan. 26, 2004. The entire contents of these applications are hereby incorporated herein by reference.

### BACKGROUND OF THE INVENTION

Obesity is a well-established risk factor for many common diseases, including diabetes, coronary heart disease, hypertension, osteoarthritis, gallbladder disease, and colon, endometrial, and breast cancer. Visceral obesity is a particularly important risk factor for these diseases. Over one in three Americans are overweight, with a substantial economic effect. Annual direct healthcare costs of disease attributable to obesity in the U.S. were estimated at approximately 52 billion dollars in 1995, or 5.7% of our national health expenditure that year. Most of the direct costs (63%) were from type II diabetes mellitus. The potential market for anti-obesity treatments exceeds $33 billion in North America annually.

There is no cure for obesity. Current methods for managing obesity include appetite suppressant drugs, drugs that block intestinal absorption of nutrients, diets, surgery, and behavioral approaches. Results of these treatments have been disappointing: only a small percentage of weight is lost and this is typically regained. Existing drug and dietary treatments for obesity are only modestly effective. Current treatments for obesity focus on dieting, surgery, and drugs. There are problems associated with each of these approaches. Many people who diet are initially successful in losing weight. However, of those who complete weight-loss programs and lose approximately 10% of their body weight, many regain two-thirds of it back within 1 year and almost all of it back within 5 years. Strict dieting alone results in loss of lean tissue as well as fat. When obese experimental animals are starved to death, some preservation of fat, despite utilization of brain and heart for energy, is observed. Surgery involves many complications and is often only used in the morbidly obese as a last resort.

Obesity is universally accompanied by a condition known as insulin resistance in which insulin is secreted at higher than normal levels in order to increase glucose consumption by tissues. Insulin resistance is defined as an impaired biological response to either exogenous or endogenous insulin. The measured biological responses could reflect metabolic processes such as changes in carbohydrate, lipid or protein metabolism as well as mitogenic processes such as alterations in growth, differentiation, DNA synthesis, and regulation of gene transcription. Insulin resistance has emerged as an important cause of glucose intolerance leading to type II diabetes mellitus and a cluster of abnormalities, including hyperlipidemia and dyslipidemia, high blood pressure, hyperuricemia, and a decrease in plasma fibrinolytic activity (Reaven, G. M. Physiol-Rev. 75(3): 473-86 (1995); “Consensus development conference on insulin resistance”, Diabetes Care, 21(2) 1998). In addition, insulin resistance can be associated with a variety of disease states such as obesity, atherosclerosis, cardiovascular syndrome X, AIDS, cancer, wasting/cachexia, sepsis, trauma associated with burns, malnutrition, lupus and other autoimmune diseases, endocrine diseases, polycystic ovary syndrome, and/or complications arising from athletic activity.

Type II diabetes mellitus is an increasingly frequent disease characterized by the inability of the body to dispose of circulating glucose. Type II Diabetes mellitus accounts for over 90% of the diagnosed cases of diabetes and affects more than 16 million people in the United States and some 200 million people around the world (Yousef et al. (1999) Diabetes Review 7:55-76). Mammals afflicted with diabetes mellitus have shown to suffer heart attacks, strokes, loss of eyesight, loss of limbs and ultimately may die as the result of this disease.

To prevent or ameliorate type II diabetes mellitus, it is necessary to prevent or reverse insulin resistance. Currently, drugs available for the treatment of diabetes mellitus act by relieving insulin resistance and enhancing glucose disposal. These drugs appear to directly or indirectly activate metabolic pathways, such as fatty acid oxidation, that generate energy for the cell in the form of ATP and NADH. It is thought that a key molecule in the action of these drugs is an enzyme known as AMPK (AMP-activated protein kinase) that controls the activity of metabolic pathways that generate energy.

AMP-activated protein kinase (AMPK) is a cytoplasmic enzyme that has been shown to exist in both the liver and skeletal muscle. AMPK acts as a “metabolic master switch”, activating ATP-producing catabolic pathways, while switching off ATP-consuming anabolic processes. It achieves this both by direct phosphorylation of metabolic enzymes, and by regulating gene expression. AMPK is activated by increasing the levels of AMP and by increasing the ratio of AMP to ATP in the cell. As the ratio of AMP:ATP increases (e.g., as levels of AMP increase or levels of ATP decrease) in a cell, AMPK is activated. Thus, when ATP consumption increases AMPK is activated, resulting in increased production of ATP to satisfy consumption.

The identification of molecules whose modulation can effect AMPK activation and, thereby effect energy generating process in cells would be of tremendous benefit.

### SUMMARY OF THE INVENTION

Adenylate kinase (AK) also plays a key role in the regulation of energy balance within cells, particularly in modulating of the ratio of AMP to ATP. Adenylate kinase is a ubiquitous enzyme that amplifies metabolic signals and promotes intracellular phosphorylation by catalyzing the reaction of ATP+AMP⇄2 ADP; Mg₂+ATP+AMP⇄Mg+ADP+ADP (Noda, The Enzymes, Vol. 8, Academic Press, Orlando Fla., 1973, pp 279-305; Atkinson Cellular Energy Metabolism and its Regulation, Academic Press, New York, 1977, pp 85-107). These two ADP molecules become substrates for ATP production in the electron transport chain.

The instant invention is based, at least in part, on the finding that inhibition of the catalytic activity of adenylate kinase results in stimulation of AMPK, enhanced production of cellular energy and enhanced fuel utilization. Accordingly, in one aspect, the invention pertains to methods for identifying an AMPK modulator, including contacting a composition comprising AK or a bioactive fragment thereof and an AK substrate or a bioactive fragment thereof with a test compound and determining the ability of the test compound to modulate interaction (e.g., binding) of AK or the AK bioactive fragment to the AK substrate or the AK substrate bioactive fragment, to thereby identify an AMPK modulator.

In other aspects, the present invention provides methods for identifying an AMPK modulator, including contacting a composition comprising AK or a bioactive fragment thereof and an AK substrate or a bioactive fragment thereof with a test compound and determining the ability of the test compound to modulate (e.g inhibit) an activity of AK or the AK bioactive fragment, to thereby identify an AMPK modulator.

In other aspects, the invention provides methods for identifying an AMPK modulator, including contacting a composition comprising or a bioactive fragment thereof and an AK substrate or a bioactive fragment thereof with a test compound and determining the ability of the test compound to modulate an activity of the substrate or the substrate bioactive fragment, to thereby identify an AMPK modulator.

In further aspects, the invention provides a method for identifying an AMPK modulator, including contacting a composition comprising AK or a bioactive fragment thereof and an AK substrate or a bioactive fragment thereof with a test compound and determining the ability of the test compound to modulate the phosphorylation state of the AK substrate or the substrate bioactive fragment, to thereby identify an AMPK modulator.

In various embodiments of the preceding aspects the modulator identified may be a positive modulator or a negative modulator of AMPK. In various other embodiments the modulator inhibits AK activity and enhances AMPK activity.

In various embodiments of the preceding aspects of the invention, the AK may be selected from the group consisting of AK1, AK2A, AK2B, AK2C, AK3, AK4 or AK5. In certain embodiments the AK is AK3. In other embodiments, the AK is AK1. The AK substrate may be AMPK or ATP. The AK bioactive fragment may be any fragment of the AK substrate having sufficient size and structure to carry out at least one activity (e.g., biological activity) of the corresponding full-length AK substrate. Exemplary bioactive fragments include, but are not limited to, enzymatic domains, protein binding and/or interaction domains, metal binding domains. The AK, AK bioactive fragment, AK substrate or the substrate bioactive fragment may be detectably labeled, radioactively labeled, or fluorescently labeled. Furthermore, in other embodiments, the interaction, activity, or phosphorylation state may be compared to an appropriate control. In addition, at least one of the AK, AK bioactive fragment, AK substrate or substrate bioactive fragment may be immobilized.

In the aspects of the present invention where the method involves a cell, the cell may overexpress the AK substrate or the bioactive fragment thereof, AK or the bioactive fragment thereof, or both the AK substrate (or substrate bioactive fragment) and AK (or AK bioactive fragment). In certain embodiments of these aspects the cell may over-express AK3. In other embodiments, the cell may over-express AK1.

In another aspect, the invention provides a modulator as identified by any of the preceding claims. The invention also provides for a pharmaceutical composition including the modulator.

In one aspect, the invention provides a method for identifying an AK modulator (e.g. an inhibitor), comprising contacting a composition comprising AMPK or a bioactive fragment thereof and an AMPK substrate or a bioactive fragment thereof with a test compound and determining the ability of the test compound to affect APMK activity in vitro.

In other aspects, the present invention provides a method for identifying an AK modulator, comprising contacting composition comprising AMPK or a bioactive fragment thereof and an AMPK substrate or a bioactive fragment thereof with a test compound and determining the ability of the test compound to affect activity of AMPK activity in the cells.

In other aspects, the invention provides methods for identifying a compound that modulates obesity, comprising contacting a cell or a composition comprising AK or a bioactive fragment thereof and an AK substrate or a bioactive fragment thereof with a test compound and determining the ability of the test compound to modulate an activity (e.g. inhibit) of AK or the AK bioactive fragment, to thereby identify an obesity modulator.

In other aspects, the invention provides a method for identifying a compound that modulates obesity, comprising contacting a cell or a composition comprising AMPK or a bioactive fragment thereof and an AMPK substrate or a bioactive fragment thereof with a test compound and determining the ability of the test compound to affect of AMPK activity in cells, to thereby identify an obesity modulator.

In other aspects, the invention provides a method for identifying a compound that modulates insulin resistance, comprising contacting a cell or a composition comprising AK or a bioactive fragment thereof and an AK substrate or a bioactive fragment thereof with a test compound and determining the ability of the test compound to modulate (e.g. inhibit) an activity of AK or the AK bioactive fragment, to thereby identify an insulin resistance modulator. In a further aspect, the invention provides, a method for identifying a compound that modulates insulin resistance, comprising contacting a cell or a composition comprising AMPK or a bioactive fragment thereof and an AMPK substrate or a bioactive fragment thereof with a test compound and determining the ability of the test compound to affect activity of AMPK activity in cells, to thereby identify an insulin resistance modulator. In various embodiments of the preceding aspects the the test compound enhances or inhibits energy consumption.

In other embodiments of the preceding aspects, the ability of the test compound to affect, for example, interaction activity or phosphorylation, includes the ability of the test compound to either enhance or inhibit such, for example, interaction activity or phosphorylation. The AK substrate may be AMPK or ATP. The AK may be AK1, AK2A, AK2B, AK2C, AK4 or AK5. In certain embodiments the AK is AK1 or AK3

In certain embodiments, the present invention provides methods of modulating energy impairment, energy consumption or energy transmission in a subject including administering to the subject an AMPK modulator identified according to the methods of any of the above methods. In particular embodiments, the method of the invention increases energy consumption or energy transmission in a subject.

In other aspects, the present invention provides methods for treating an energy related disease or disorder (e.g., type II diabetes, insulin resistance or obesity) including administering any of the pharmaceutical compositions described above.

### BRIEF DESCRIPTION OF DRAWINGS

FIG. 1 depicts the adenylate kinase pathway to AMPK activation. Inhibition of adenylate kinase leads to an increase in the AMP: ATP ratio, stimulation of AMPK, and stimulation of fuel utilization which will be beneficial in increasing energy consumption and in ameliorating insulin resistance.

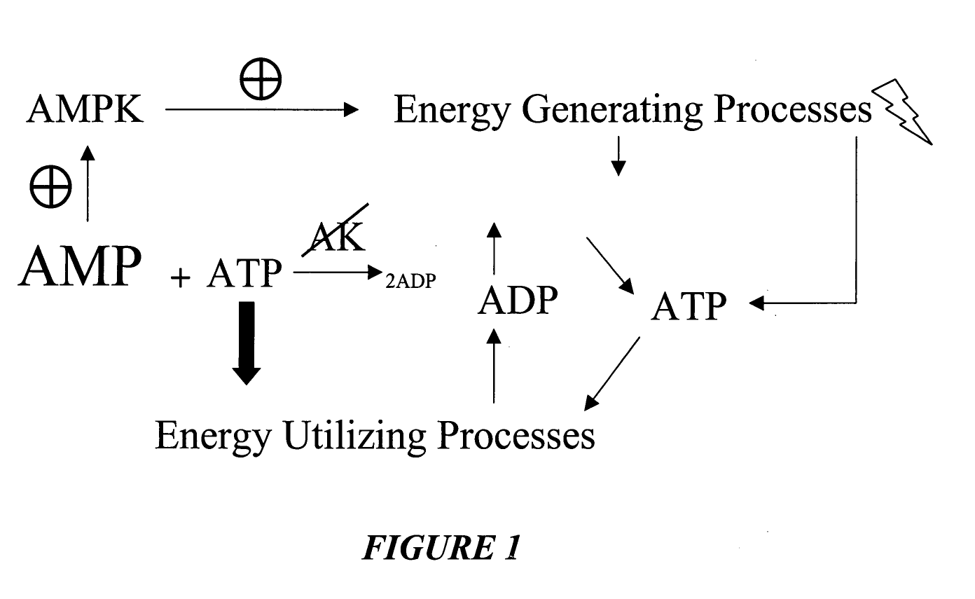

### DETAILED DESCRIPTION OF THE INVENTION

The present invention is based, at least in part, on the finding that inhibition of the catalytic activity of adenylate kinase results in stimulation of AMPK, enhanced production of cellular energy and enhanced fuel utilization. In general, adenylate kinases play a key role in the regulation of energy balance within cells, particularly maintenance of the ratio of ATP with its diphosphate (ADP) and monophosphate forms (AMP). When ATP is depleted and ADP accumulates, two molecules of ADP can dismutate by the reverse action to form ATP. When AMP concentrations are relatively high, ADP can be formed from AMP (and ATP); the ADP can then undergo phosphorylation to re-form ATP. AMPK is a downstream component of a protein kinase cascade that is switched on by the rise in the AMP:ATP ratio. In this manner, the activity of adenylate kinase affects the mechanism of AMPK function. Once activated, the AMPK cascade switches on catabolic pathways that generate ATP, while switching off ATP-consuming processes that are not essential for short term cell survival (e.g., lipid, carbohydrate, or protein synthesis). (Hardie. 2003. Endocrinology 144:5179). By reducing the activity of AK, AMPK is activated and cells burn more fuel (e.g., fat) to generate the same amount of ATP. The present invention also provides methods of identifying modulators of AK or AMPK and methods of treating an energy related disease or disorder e.g., obesity or type II diabetes.

So that the invention may be more readily understood, certain terms are first defined.

I. Definitions

As used herein, the term “contacting” (i.e., contacting a cell e.g. an immune cell, an adipocyte, with an compound) is intended to include incubating the compound and the cell together in vitro (e.g., adding the compound to cells in culture) or administering the compound to a subject such that the compound and cells of the subject are contacted in vivo. The term “contacting” is not intended to include exposure of cells to a modulator or compound that may occur naturally in a subject (i.e., exposure that may occur as a result of a natural physiological process).

As used herein, the term “modulate” means to change, affect or interfere with the functioning of the components of these kinase enzyme systems, e.g., the enzymatic activity. For example, the modulation can be the inhibition of or enhancement of enzymatic activity.

As used herein, the term “test compound” includes a compound that has not previously been identified as, or recognized to be a modulator of the activity of AK or a modulator of AMPK.

The term “library of test compounds” is intended to refer to a panel comprising a multiplicity of test compounds.

As used herein, the term “cell free composition” refers to an-isolated composition which does not contain intact cells. Examples of cell free compositions include cell extracts and compositions containing isolated proteins.

AMP-activated protein kinase (AMPK) was discovered at approximately the same time as cAMP-dependent protein kinase, but only recently have important regulatory functions relating to diabetes been elucidated. Winder, W W & Hardie, D G Am J Physiol 277:E1-E10 (1999). AMPK molecules are heterotrimeric complexes comprised of a catalytic alpha subunit and two non-catalytic beta and gamma subunits. The alpha subunit comprises the kinase domain at the N and C-terminus. The beta domain comprises the glycogen binding domain. The gamma subunit comprises four tandem repeats of a motif known as a CBS domain. Hardie, D G & Carling, D Eur J Biochem 246:259-273 (1997); Hardie, D G et al. Ann Rev Biochem 67:821-855 (1998). This kinase is activated by both phosphorylation and allosteric mechanisms. It can be phosphorylated and activated by an upstream kinase, AMPKK, and is also activated allosterically by increases in the 5′-AMP/ATP ratio. In muscle, creatine phosphate (CP) allosterically inhibits the enzyme. Ponticos, M et al. EMBO J 17:1688-1699 (1998). In liver, AMPK plays the role of phosphorylating and inactivating acetyl-CoA carboxylase (ACC) and 3-hydroxy-3-methylglutaryl-CoA reductase (HMGR), the rate limiting enzymes of fatty acid and cholesterol biosynthesis.

AMPK is activated in skeletal muscle of rats during treadmill running and in response to electrical stimulation. Winder, W W & Hardie, D G Am J Physiol 270:E299-E304 (1996); Hutber, C A et al. Am J Physiol 272:E262-E266 (1997); Vavvas, D et al. J Biol. Chem 272:13256-13261 (1997); Ihlemann, J et al. Am J Physiol 277:E208-E214 (1999); Rasmussen, B B & Winder, W W J Appl Physiol 83:1104-1109 (1997). As muscle contracts, ATP is used as a source of energy, generating ADP and inorganic phosphate. The ADP can be phosphorylated to form ATP in the glycolytic pathway in the sarcoplasm or by oxidative phosphorylation in the mitochondria. Non-oxidative, rapidly acting mechanisms also include the action of myokinase, which makes one ATP and one 5′-AMP from two ADP molecules, and the action of creatine phosphokinase, which transfers a phosphate from CP to ADP forming ATP. These changes occur rapidly at the beginning of muscle contraction resulting in a drop in CP, the allosteric inhibitor of AMPK, and an increase in 5′-AMP, the allosteric activator of AMPKK and AMPK.

The term “AMPK” includes naturally occurring AMPK molecules (e.g., mammalian, such as human AMPK molecules) and their equivalents.

The phrase “modulator of AMP-activated protein kinase” (AMPK) includes molecules which can modulate the expression and/or activity of AMPK, e.g., the ability of the kinase to act on a substrate (e.g., to phosphorylate the substrate) or the ability of the kinase to modulate the balance between energy generation and energy consumption.Modulators of AMPK may act on AMPK directly or may modulate AMPK indirectly, e.g., by acting on a molecule in an energy pathway involving AMPK, e.g., AK. Exemplary modulators of AMPK include e.g., modulators of AK1, AK2A, AK2B, AK2C, AK3, AK4 and/or AK5. In certain embodiments, the modulators of AMPK stimulate AMPK activity, e.g. inhibit AK activity. Preferred modulators include inhibitors of AK1 and/or AK3.

The phrase “AMPK substrate” refers to a molecule which is phosphorylated by AMPK on a serine or threonine residue. Preferred AMPK substrates include Acetyl-CoA carboxylase, Casein, Glycogen synthase, HMG-CoA reductase, Hormon sensitive lipase, Phosphorylase kinase and IRS1 (Carling & Hardie, 1989 BBA 1012:81-86; Jacobsen et al., 2001 JBC 276:46912-46916).

Adenylate kinase (AK, ATP: AMP phosphotransferase) is a ubiquitous enzyme that catalyses the interconversion of three adenine nucleotides in the cell; Mg₂+ATP+AMP⇄Mg₂+ADP+ADP (Noda, The Enzymes, vol. 8, Academic Press, Orlando, Fla., 1973, pp 279-305; Atkinson Cellular energy metabolism and its regulation, Academic Press, New York, 1977, pp 85-107). In vertebrates, five isozymes (AK1, AK2, AK3, AK4 and AK5) have been identified to date. The term “AK” includes naturally occurring AK molecules (e.g., mammalian, such as human AK molecules) and their equivalents.

The term “adenylate kinase” as used herein includes the various AK isozymes, e.g., AK1 (NCBI Accession No.: NM000476) (NCBI Accession No. NP000467) (or myokinase), which is a cytosolic enzyme present in brain, skeletal muscle, and erythrocytes, and AK2 (NCBI Accession Nos. NM001625, NM 013411, NM172199) (NCBI Accession No. NP01616, NP37543, NP751949), which is associated with the mitochondrial membrane in liver, spleen, heart, and kidney, both utilize ATP as their nucleoside triphosphate donor substrate. AK3 (NCBI Accession Nos. NM 13410, NP0377542)(NCBI Accession Nos. NM16282, NP 057366) (or GTP:AMP phosphotransferase) is located in the mitochondrial matrix, primarily in heart and liver cells, and uses MgGTP instead of MgATP. AK4 (NCBI Accession No. NM 017135)(NCBI Accession No. NP058831) and AK5 (NCBI Accession Nos. NM0174858, NM 012093)(NCBI Accession Nos NP777283, NP036225) are both localized in brain tissue. AK molecules comprise an AMP binding domain, a core domain and two peripheral domains involved in covering the ATP active site. Several regions of AK family enzymes are well conserved, including the nucleoside triphosphate binding glycine-rich region, the nucleoside monophosphate binding site, and the lid domain that closes over the substrate upon binding (see Schulz (I 987) Cold Spring Harbor Symp. Quant. Biol. 52:429-439). Other mammalian homologs (e.g. rodents) of these exemplary AK enzymes are also included in the term and can be obtained readily using standard procedures.

The phrase “modulator of adenylate kinase” (AK) includes molecules which can modulate the expression and/or activity of AK, e.g., the ability of the kinase to act on a substrate (e.g., to phosphorylate the substrate) or the ability of the kinase to modulate the balace between energy generation and energy consumption in a cell. Modulators of AK may act on AK directly or may modulate AK indirectly, e.g., by acting on a molecule in an energy pathway involving AK.

The phrase “AK substrate” refers to a substance on which AK acts catalytically. Preferred AMPK substrates include AMP and ATP.

The phrase “energy related state” or “energy related disease or disorder” includes diseases or conditions which would benefit from modulation of the balance between the generation of and the consumption of energy in a cell. Suitable examples of energy related states include obesity, insulin dependent diabetes mellitus i.e., type II diabetes and its related disorders, mitochondrial related diseases, wasting syndromes.

The term “energy impairment” includes those states characterized by impaired energy generation. The term “energy generation” includes the generation of energy usable by a cell, e.g., in the form of ATP. The term “energy consumption” includes the utilization of energy by a cell, e.g., in the form of ATP, in a metabolic process.

As used herein, a “naturally-occurring” nucleic acid molecule refers to an RNA or DNA molecule having a nucleotide sequence that occurs in nature (e.g., encodes a natural protein).

The term “equivalent” is intended to include nucleotide sequences encoding polypeptides that are functionally equivalent (e.g., to AK or AMPK) i.e., proteins which are structurally related and maintain at least one biological activity of the native nucleic acid molecule.

As used herein, the term “dominant negative” includes polypeptide molecules (e.g., portions or variants thereof) that compete with native (i.e., wild-type) polypeptide molecules, but which compete with the native polypeptide and lack at least one activity of the native polypeptide, thereby downmodulating the activity of the native polypeptide.

As used herein, the term “nucleic acid molecule” includes DNA molecules (e.g., cDNA or genomic DNA) and RNA molecules (e.g., mRNA). The nucleic acid molecule may be single-stranded or double-stranded, but preferably is double-stranded DNA.

As used herein, a “naturally-occurring” nucleic acid molecule refers to an RNA or DNA molecule having a nucleotide sequence that occurs in nature (e.g., encodes a natural protein).

An used herein, an “isolated nucleic acid molecule” refers to a nucleic acid molecule that is free of gene sequences which naturally flank the nucleic acid in the genomic DNA of the organism from which the nucleic acid is derived (i.e., gene sequences that are located adjacent to the isolated nucleic molecule in the genomic DNA of the organism from which the nucleic acid is derived). For example, in various embodiments, an isolated AK or AMPK nucleic acid molecule may contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, may be free of other cellular material.

As used herein, the term “hybridizes under stringent conditions” is intended to describe conditions for hybridization and washing under which nucleotide sequences at least 60% homologous to each other typically remain hybridized to each other. Preferably, the conditions are such that at least sequences at least 65%, more preferably at least 70%, and even more preferably at least 75% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

As used herein, an “antisense” nucleic acid molecule comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

In one embodiment, a nucleic acid molecule of the invention is a compound that mediates RNA interference, RNAi. RNA interfering agents include, but are not limited to, nucleic acid molecules including RNA molecules which are homologous to the target gene or genomic sequence, e.g., AK or AMPK, or a fragment thereof, “short interfering RNA” (siRNA), “short hairpin” or “small hairpin RNA” (shRNA), and small molecules which interfere with or inhibit expression of a target gene by RNA inerference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries known in the art for use in antisense RNA can be employed.

As used herein, an “isolated protein” or “isolated polypeptide” refers to a protein or polypeptide that is substantially free of-other proteins, polypeptides, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the protein is derived, or substantially free from chemical precursors or other chemicals when chemically synthesized. The language “substantially free of cellular material” includes preparations of, for example, AK or AMPK, protein in which the protein is separated from cellular components of the cells from which it is isolated or recombinantly produced.

The nucleic acids of the invention can be prepared by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “host cell” refers to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “transgenic cell” refers to a cell containing a transgene.

As used herein, a “transgenic animal” refers to a non-human animal, e.g., a swine, a monkey, a goat, or a rodent, e.g., a mouse, in which one or more, and preferably essentially all, of the cells of the animal include a transgene, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

As used herein, the term “rodent” refers to all members of the phylogenetic order Rodentia.

As used herein, a “homologous recombinant animal” refers to a type of transgenic non-human animal, preferably a mammal, more preferably a mouse, in which an endogenous gene, e.g., AK or AMPK, has been altered by homologous recombination between the endogenous gene and an exogenous DNA molecule introduced into a cell of the animal, e.g., an embryonic cell of the animal, prior to development of the animal.

As used herein, the term “misexpression” includes a non-wild-type pattern of gene expression. Expression as used herein includes transcriptional, post transcriptional, e.g., mRNA stability, translational, and post translational stages. Misexpression includes: expression at non-wild-type levels, i.e., over or under expression; a pattern of expression that differs from wild-type in terms of the time or stage at which the gene is expressed, e.g., increased or decreased expression (as compared with wild-type) at a predetermined developmental period or stage; a pattern of expression that differs from wild-type in terms of decreased expression (as compared with wild-type) in a predetermined cell type or tissue type; a pattern of expression that differs from wild-type in terms of the splicing size, amino acid sequence, post-translational modification, or biological activity of the expressed polypeptide; a pattern of expression that differs from wild-type in terms of the effect of an environmental stimulus or extracellular stimulus on expression of the gene, e.g., a pattern of increased or decreased expression (as compared with wild-type) in the presence of an increase or decrease in the strength of the stimulus. 5 Misexpression includes any expression from a transgenic nucleic acid. Misexpression includes the lack or non-expression of a gene or transgene, e.g., that can be induced by a deletion of all or part of the gene or its control sequences.

As used herein, the term “knockout” refers to an animal or cell therefrom, in which the insertion of a transgene disrupts an endogenous gene in the animal or cell therefrom. For example, the disruption can essentially eliminate AK in the animal or cell. In preferred embodiments, misexpression of the gene encoding the AK is caused by disruption of the gene encoding AK. For example, the gene can be-disrupted through removal of DNA encoding all or part of the protein.

In preferred embodiments, the animal can be heterozygous or homozygous for a misexpressed gene, e.g., it can be a transgenic animal heterozygous or homozygous for a transgene encoding AK or AMPK.

In preferred embodiments, the animal is a transgenic mouse with a transgenic disruption of the gene encoding AK (e.g AK1 or AK3), preferably an insertion or deletion, which inactivates the gene product.

In another aspect, the invention features, a nucleic acid molecule which, when introduced into an animal or cell, results in the misexpression of the AK gene in the animal or cell. In certain preferred embodiments, introduction of the nucleic acid molecule results in misexpression of the AK1 or AK3 gene In other preferred embodiments, the nucleic acid molecule, includes an AK nucleotide sequence which includes a disruption, e.g., an insertion or deletion and preferably the insertion of a marker sequence.

As used herein, the term “marker sequence” includes a nucleic acid molecule that (a) is used as part of a nucleic acid construct (e.g., the targeting construct) to disrupt the expression of the gene of interest (e.g., AK) and (b) is used to identify those cells that have incorporated the targeting construct into their genome. For example, the marker sequence can be a sequence encoding a protein which confers a detectable trait on the cell, such as an antibiotic resistance gene, e.g., neomycin resistance gene, or an assayable enzyme not typically found in the cell, e.g., alkaline phosphatase, horseradish peroxidase, luciferase, beta-galactosidase and the like.

As used herein, “disruption of a gene” refers to a change in the gene sequence, e.g., a change in the coding region. Disruption includes: insertions, deletions, point mutations, and rearrangements, e.g., inversions. The disruption can occur in a region of the native AK DNA sequence (e.g., one or more exons) and/or the promoter region of the gene so as to decrease or prevent expression of the gene in a cell as compared to the wild-type or naturally occurring sequence of the gene. The “disruption” can be induced by classical random mutation or by site directed methods. Disruptions can be transgenically introduced. The deletion of an entire gene is a disruption. Preferred disruptions reduce AK levels to about 50% of wild-type, in heterozygotes or essentially eliminate AK in homozygotes.

In one embodiment, small molecules may be used as test compounds. The term “small molecule” is a term of the art and includes molecules that are less than about 7500, less than about 5000, less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., Cane et al. 1998. Science 282:63), and natural product extract libraries. In another embodiment, the compounds are small, organic non-peptidic compounds. In a further embodiment, a small molecule is not biosynthetic. For example, a small molecule is preferably not itself the product of transcription or translation.

Various aspects of the invention are described in further detail in the following subsections:

II. Screening Assays

According to the invention, the following assays may be used to identify compounds that modulate interaction (e.g., binding) of AK or bioactive fragments thereof with AK substrates or bioactive fragments thereof. These modulators of AK act as AMPK modulators. Such AMPK modulators are particularly useful in regulation of the balance between energy generation and energy consumption. The assays feature identifying modulators of the expression and/or activity of AK or bioactive fragments thereof. In certain embodiments, the assays of the present invention feature identifying compounds that modulate the phosphorylation state of AK substrates. In other embodiments, the invention features methods for identifying AK modulators using compositions comprising AMPK or bioactive fragments thereof. In further embodiments the assays feature identifying compounds that inhibit AK activity. In still other embodiments the AK modulators identified according to the methods of the invention modulate (e.g. inhibit) AK1 or AK3.

The subject screening assays employ indicator compositions. These indicator compositions comprise the components required for performing an assay that detects and/or measures a particular event. The indicator compositions of the invention provide a reference readout and changes in the readout can be monitored in the presence of one or more test compounds. A difference in the readout in the presence and the absence of the compound indicates that the test compound is a modulator of the molecule(s) present in the indicator composition.

The indicator composition used in the screening assay can be a cell that expresses an AK polypeptide and/or an AMPK polypeptide. For example, a cell that naturally expresses or, more preferably, a cell that has been engineered to express one or more of the proteins by introducing into the cell an expression vector encoding the one or more of the proteins may be used. Preferably, the cell is a mammalian cell, e.g., a human cell. In one embodiment, the cell is an adipocyte. Alternatively, the indicator composition can be a cell-free composition that includes one or more of the proteins (e.g., a cell extract or a composition that includes e.g., either purified natural or recombinant protein).

Compounds that modulate the expression and/or activity of AK and/or AMPK identified using the assays described herein can be useful for treating a subject that would benefit from the modulation of the balance between energy generation and energy consumption in cells. Exemplary conditions include metabolic disorders (e.g., obesity or wasting syndromes) and insulin resistance.

The subject screening assays can be performed in the presence or absence of other agents. In one embodiment, the subject assays are performed in the presence of an agent that simulates conditions of exercise, e.g., tunicamycin, or other molecules that induce stress.

In one embodiment, secondary assays can be used to confirm that the modulating agent effects an AK molecule or an AMPK molecule. For example, compounds identified in a primary screening assay can be used in a secondary screening assay to determine whether the compound affects the balance between energy consumption and energy generation. For example, a modulating agent can be identified using a cell-based or a cell-free assay, and the ability of the agent to modulate the activity of AK or AMPK can be confirmed in vivo, e.g., in an animal such as, for example, in an animal model of type II diabetes or obesity.

Moreover, a modulator as described herein (e.g., an antisense nucleic acid molecule or a small molecule) may be used in an animal model to determine the efficacy, toxicity, or side effects of treatment with such a modulator. Alternatively, a modulator identified as described herein may be used in an animal model to determine the mechanism of action of such a modulator.

In one embodiment, the screening assays of the invention are high throughput or ultra high throughput (e.g., Fernandes P B, Curr Opin Chem Biol. 1998 2:597; Sundberg S A, Curr Opin Biotechnol. 2000, 11:47).

Exemplary cell based and cell free assays of the invention are described in more detail below.

A. Cell Free Assays

In one embodiment, the indicator composition can be a cell-free composition that includes an AK(e.g. AK1 or AK3) and/or AMPK e.g., a cell extract from a cell expressing the protein or a composition that includes purified either natural or recombinant protein. Polypeptides expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies may be used to produce a purified or semi-purified protein that may be used in a cell free composition. Alternatively, a lysate or an extract of cells expressing the protein of interest can be prepared for use as cell-free composition. Cell extracts with the appropriate post-translation modifications of proteins can be prepared using commercially available resources found at, for example Promega, Inc., and include but are not limited to reticulocyte lysate, wheat germ extract and E. coli S30 extract.

In one embodiment, compounds that specifically modulate an activity of AK or AMPK may be identified. In one embodiment, to measure the activity of AK, the change in the amounts of adenine nucleotides brought about in a measured interval of time by can be determined after separation of the nucleotides by chromatographic procedures. An example of such a method is the use of Dowex-1 resin with electrophoretic techniques using thin layer chromatography and chromatography paper (Sato et. al., Anal Biochem. 5, 542, ; Randerath, Nature, 194, 768, 1962; Krebs et. al., BBA 12, 172, 1953).

In another embodiment, a reaction catalyzed by AK can be coupled to one catalyzed by another enzyme, e.g., creatine kinase. In the case of creatine kinase excess creatine is added together with creatine kinase to yield creatine phosphate from the ATP formed by adenylate kinase. Another widely used coupled reaction is the use of adenylate kinase and ATP coupled with excess phosphoenolpyruvate together with lactate dehydrogenase and excess DPNH. The decrease in DPNH can be measured by the decrease in absorbence at 340 nm with time (Oliver B J 61, 116,1955; Adams Biochem. Z. 335, 25,1961). A pH-stat assay couples the reaction of adenylate kinase with ADP as substrate with the hexokinase reaction carried out at pH 8, in which one mole of hydrogen ions is released for every mole of ATP formed by adenylate kinase. The rate to which standardized alkali is added to maintain the pH is a measure of adenylate kinase activity.

In another embodiment, AK activity can be determined by utilizing labeled substrates that can be separated and visualized on sensitive films.

AMPK activity may be assayed using the purified catalytic subunit to phosphorylate target proteins. Intracellular targets include ion channels, transcriptional activator proteins and regulatory enzymes involved in metabolism. In addition, the regulatory subunit is useful for confirming the phosphorylation arising from AMPK. Inhibition of phosphorylation by addition of R₂, and reversal of inhibition by subsequent addition cAMP, provides definitive evidence that phosphorylation is catalyzed by AMPK.

In one embodiment, AMPK activity can be assayed using 32P-labelled ATP and the SAMS peptide, as described by Davies et al., Eur. J. Biochem., 186:123-128 (1989).

In the methods of the invention for identifying test compounds that modulate an interaction between a AK or AMPK and a substrate or upstream activating molecule, the complete protein may be used in the method, or, alternatively, biologically active portions of the protein may be used. An assay may be used to identify test compounds that either stimulate or inhibit the interaction between the protein and a upstream activating molecule or substrate.

In one embodiment, the amount of binding of AK or AMPK to an upstream activating molecule or a substrate in the presence of the test compound is greater than the amount of binding in the absence of the test compound, in which case the test compound is identified as a compound that enhances binding of AK or AMPK to an upstream activating molecule or a substrate. In another embodiment, the amount of binding of the AK or AMPK to an upstream activating molecule or a substrate in the presence of the test compound is less than the amount of binding of AK or AMPK to an upstream activating molecule or a substrate in the absence of the test compound, in which case the test compound is identified as a compound that inhibits binding of AK or AMPK to an upstream activating molecule or a substrate.

For example, binding of an upstream activating molecule or a substrate to AK or AMPK can be determined either directly or indirectly as described above. Binding can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) may be used as an indication of real-time reactions between biological molecules.

In another embodiment, the ability of a compound to modulate the ability of AK or AMPK to be acted on by an enzyme or to act on a substrate can be measured.

In one embodiment of the above assay methods, it may be desirable to immobilize either one or more of the reactants, for example, to facilitate separation of complexed from uncomplexed forms of one or both of the proteins, or to accommodate automation of the assay. Binding to a surface can be accomplished in any vessel suitable for containing the reactants. Examples of such vessels include microtitre plates, test tubes, and micro-centrifuge tubes. In one embodiment, a fusion protein can be provided in which a domain that allows one or both of the proteins to be bound to a matrix is added to one or more of the molecules. For example, glutathione-S-transferase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or AK protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of binding or activity determined using standard techniques.

Other techniques for immobilizing proteins on matrices can also be used in the screening assays of the invention. For example, proteins may be immobilized utilizing conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with protein or target molecules but which do not interfere with binding of the protein to its target molecule can be derivatized to the wells of the plate, and unbound target or AK or AMPK protein is trapped in the wells by antibody conjugation. Methods for detecting such complexes, in addition to those described above for the GST-immobilized complexes, include immunodetection of complexes using antibodies reactive with AK or AMPK or target molecule, as well as enzyme-linked assays which rely on detecting an enzymatic activity associated with AK or AMPK.

In another embodiment, the assay is a cell-free assay in which a composition comprising an AK polypeptide and an AK substrate polypeptide (or bioactive portions thereof) is contacted with a test compound and the ability of the test compound to modulate (e.g., stimulate or inhibit) the activity of the AK polypeptide or AK substrate polypeptide (or biologically active portions thereof) is determined.

Determining the ability-of the test compound to modulate the activity of an AK or an AK substrate polypeptide can be accomplished, for example, by determining the ability of the AK substrate polypeptide to modulate the activity of a downstream binding partner or target molecule by one of the methods described herein for cell-based assays. For example, the catalytic/enzymatic activity of the target molecule on an appropriate downstream substrate can be determined.

B. Cell Based Assays

The indicator compositions of the invention may be cells that express AK or AMPK. For example, a cell that naturally expresses endogenous polypeptide, or, more preferably, a cell that has been engineered to express one or more exogenous polypeptides, e.g., by introducing into the cell an expression vector encoding the protein may be used in a cell based assay.

The cells used in the instant assays can be eukaryotic or prokaryotic in origin. For example, in one embodiment, the cell is a bacterial cell. In another embodiment, the cell is a fungal cell, e.g., a yeast cell. In another embodiment, the cell is a vertebrate cell, e.g., an avian or a mammalian cell (e.g., a murine cell, or a human cell). In a preferred embodiment, the cell is a human cell.

Recombinant expression vectors that may be used for expression of polypeptides are known in the art. For example, the cDNA is first introduced into a recombinant expression vector using standard molecular biology techniques. A cDNA can be obtained, for example, by amplification using the polymerase chain reaction (PCR) or by screening an appropriate cDNA library.

Following isolation or amplification of a cDNA molecule encoding the gene of interest, e.g., AK or AMPK, or a biologically active fragment thereof the DNA fragment is introduced into an expression vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g.;-bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule in a form suitable for expression of the nucleic acid in a host cell, which means that the recombinant expression vectors include one or more regulatory sequences, selected on the basis of the host cells to be used for expression and the level of expression desired, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

When used in mammalian cells, the expression vector's control functions are often provided by viral regulatory elements. For example, commonly used promoters are derived from polyoma virus, adenovirus, cytomegalovirus and Simian Virus 40. Non-limiting examples of mammalian expression vectors include pCDM8 (Seed, B., (1987) Nature 329:840) and pMT2PC (Kaufmnan et al. (1987), EMBO J. 6:187-195). A variety: of mammalian expression vectors carrying different regulatory sequences are commercially available. For constitutive expression of the nucleic acid in a mammalian host cell, a preferred regulatory element is the cytomegalovirus promoter/enhancer. Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Still further, many tissue-specific regulatory sequences are known in the art, including the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Baneiji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916) and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Vector DNA may be introduced into mammalian cells via conventional transfection techniques. As used herein, the various forms of the term “transfection” are intended to refer to a variety of art-recognized techniques for introducing foreign nucleic acid (e.g., DNA) into mammalian host cells, including calcium phosphate co-precipitation, DEAE-dextran-mediated transfection, lipofection, or electroporation. Suitable methods for transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on a separate vector from that encoding, e.g., AK or AMPK.

Cells stably transfected with the introduced nucleic acid can be identified by drug selection (e.g., cells that have incorporated the selectable marker gene will survive, while the other cells die).

In one embodiment, within the expression vector coding sequences are operatively linked to regulatory sequences that allow for constitutive expression of the molecule in the indicator cell (e.g., viral regulatory sequences, such as a cytomegalovirus promoter/enhancer, may be used). Use of a recombinant expression vector that allows for constitutive expression of the genes in the indicator cell is preferred for identification of compounds that enhance or inhibit the activity of the molecule. In an alternative embodiment, within the expression vector the coding sequences are operatively linked to regulatory sequences of the endogenous gene (i.e., the promoter regulatory region derived from the endogenous gene). Use of a recombinant expression vector in which expression is controlled by the endogenous regulatory sequences is preferred for identification of compounds that enhance or inhibit the transcriptional expression of the molecule.

The ability of the test compound to modulate AK or AMPK binding to a substrate or upstream activating molecule can also be determined. This can be accomplished, for example, by determining the ability of the molecules to be coimmunoprecipitated or by coupling the binding molecule with a radioisotope or enzymatic label such that binding of the target molecule to AK or AMPK can be determined, e.g., by detecting the labeled molecule in a complex.

Determining the ability of the test compound to bind can be accomplished, for example, by coupling the compound with a radioisotope or enzymatic label such that binding of the compound can be determined by detecting the labeled compound in a complex. For example, targets can be labeled with ¹²⁵I, ³⁵S, ¹⁴C, or ³H, either directly, or indirectly, and the radioisotope detected by direct counting of radioemmission or by scintillation counting. Alternatively, compounds can be labeled, e.g., with, for example, horseradish peroxidase, alkaline phosphatase, or luciferase, and the enzymatic label detected by determination of conversion of an appropriate substrate to product.

In another embodiment, fluorescence technologies can be used, e.g., fluorescence polarization, time-resolved fluorescence, and fluorescence resonance energy transfer (Selvin P R, Nat. Struct. Biol. 2000 7:730; Hertzberg R P and Pope A J, Crurr Opin Chem Biol. 2000 4:445).

It is also within the scope of this invention to determine the ability of a compound to interact with AK or AMPK without the labeling of any of the interactants. For example, a microphysiometer may be used to detect the interaction of a binding molecule with AK or AMPK without the labeling of either the compound or the molecule (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate may be used as an indicator of the interaction between compounds.

In one aspect of the invention, the AK or AMPK polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with AK or AMPK (“binding proteins” or “target molecules”). Such target molecules are also likely to be involved in the regulation of cellular activities modulated by AK or AMPK. Determining the ability of the test compound to modulate the activity of an AK substrate or AK polypeptide (or biologically active portions thereof) can be accomplished by assaying for any of the activities of an AK substrate or AK polypeptide described herein.

Determining the ability of the test compound to modulate the activity of an AK substrate polypeptide or AK polypeptide (or biologically active portions thereof) can also be accomplished by assaying for the activity of an AK substrate target molecule. In one embodiment, determining the ability of the test compound to modulate the activity of an AK substrate polypeptide, or biologically active portion thereof, is accomplished by assaying for the ability to bind AK or a bioactive portion thereof.

In some embodiments, the cell overexpresses the AK or AMPK substrate polypeptide, or biologically active portion thereof, and optionally, overexpresses AK or AMPK, or biologically active portion thereof. In certain embodiments, the cell overexpresses AK1 or AK3.

C. Assays Using Knock-Out Cells

In another embodiment, the invention provides methods for identifying compounds that modulate the balance between energy generation and energy production in cells deficient in AK or AMPK. As described in the Examples, inhibition of AK in cells results in activation of AMPK and increased consumption of fuel to generate the same amount of energy. Thus, cells deficient in AK or AMPK may be used identify agents that modulate the balance between energy consumption and energy generation. Alternatively, a “conditional knock-out” system, in which the gene is rendered non-functional in a conditional manner, may be used to create deficient cells for use in screening assays. For example, a tetracycline-regulated system for conditional disruption of a gene as described in WO 94/29442 and U.S. Pat. No. 5,650,298 may be used to create cells, or animals from which cells can be isolated, deficient in specific polypeptides in a controlled manner through modulation of the tetracycline concentration in contact with the cells.

Methods for making cells deficient in-AK are known in the art (Janssen et al. (2000), The EMBO Journal, Vol.19, No.23; pp 6371-6381). Certain preferred cells are deficient in AK1 or AK3.

In one embodiment, the test compound is administered directly to a non-human knock out animal, preferably a mouse (e.g., a mouse in which the AK or AMPK) is conditionally disrupted by means described above, or a chimeric mouse in which the lymphoid organs are deficient in the gene, to identify a test compound that modulates the in vivo responses of cells deficient in the gene. In another embodiment, cells deficient in the gene are isolated from the non-human animal and contacted with the test compound ex vivo to identify a test compound that modulates a response regulated by the gene in the cells

Preferred non-human animals include monkeys, dogs, cats, mice, rats, cows, horses, goats and sheep. In preferred embodiments, the deficient animal is a mouse. Mice deficient in the gene can be made using methods known in the art. Non-human animals deficient in a particular gene product typically are created by homologous recombination. In an exemplary embodiment, a vector is prepared which contains at least a portion of the gene into which a deletion, addition or substitution has been introduced to thereby alter, e.g., functionally disrupt, the endogenous gene. The gene preferably is a mouse gene. For example, a mouse gene can be isolated from a mouse genomic DNA library using the mouse cDNA as a probe. The mouse gene then may be used to construct a homologous recombination vector suitable for modulating an endogenous gene in the mouse genome. In a preferred embodiment, the vector is designed such that, upon homologous recombination, the endogenous gene is functionally disrupted (i.e., no longer encodes a functional protein; also referred to as a “knock out” vector).

Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced gene has homologously recombined with the endogenous gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells may be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In one embodiment, compounds that modulate energy generation versus energy consumption are identified by contacting cells deficient in AK or AMPK are contacted with one or more test compounds ex vivo with the compound and determining the effect of the test compound on a read-out.

In one embodiment of the screening assay, compounds are contacted with deficient cells by administering the test compound to a non-human deficient animal in vivo and evaluating the effect of the test compound on the response in the animal.

The test compound can be administered to a non-knock out animal as a pharmaceutical composition. Such compositions typically comprise the test compound and a pharmaceutically acceptable carrier. As used herein the term “pharmaceutically acceptable carrier” includes any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions. Pharmaceutical compositions are described in more detail below.

Following contact of the deficient cells with a test compound (either ex vivo or in vivo), the effect of the test compound on the balance between energy consumption and energy generation can be determined by any one of a variety of suitable methods, such as those set forth herein.

III. Test Compounds

A variety of test compounds can be evaluated using the screening assays described herein. The term “test compound” includes any reagent or test agent which is employed in the assays of the invention and assayed for its ability to influence the expression and/or activity of AK or AMPK, or a downstream effect of modulation of AK or AMPK. More than one compound, e.g., a plurality of compounds, can be tested at the same time for their modulatory ability. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening may be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303-), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061-).

The compounds of the present invention can be obtained using any of the numerous approaches for generating a compound libraries known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al. (1996) Immunopharmacology 33:68-; and in Gallop et al. (1994); J. Med. Chem. 37:1233.

Exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., antibodies (e.g., intracellular, polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), and 6) mutant forms of molecules (e.g,; dominant negative mutant forms of AK or AMPK).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al (1994) J Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In a preferred embodiment, the library is a natural product library.

Compounds identified in the subject screening assays may be used, e.g., in methods of modulating the balance of energy consumption and energy generation. It will be understood that it may be desirable to formulate such compound(s) as pharmaceutical compositions (described supra) prior to contacting them with cells.

Once a test compound is identified that directly or indirectly modulates, e.g., the activity of AK (e.g. AK1 or AK3) or AMPK, the selected test compound (or “compound of interest”) can then be further evaluated for its effect on cells, for example by contacting the compound of interest with cells either in vivo (e.g., by administering the compound-of interest to a subject) or ex vivo (e.g., by isolating cells from the subject and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

The instant invention also pertains to compounds identified in the subject screening assays.

IV. Pharmaceutical Compositions

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will preferably be sterile and should be fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

In one embodiment, the test compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

V. Methods of Use

The present invention provides for both prophylactic and therapeutic methods of treating a subject at risk of (or susceptible to) a disorder or having a disorder that would benefit from modulation of AK or AMPK. Such disorders include those associated with an aberrant balance between energy consumption and energy generation. For example, obesity, wasting disorders, or type II diabetes.

In one aspect, the invention provides a method for preventing in a subject, a disease or condition associated with an aberrant balance between energy consumption and energy generation, by administering to the subject an agent which modulates the activity of AK or AMPK. Subjects at risk for such disorders can be identified, for example, using methods described herein or any one or a combination of diagnostic or prognostic assays known in the art. Administration of a prophylactic agent can occur prior to the manifestation of symptoms characteristic of the aberrant balance between energy consumption and energy generation, such that a disease or disorder is prevented or, alternatively, delayed-in its progression. Agents for use can beknown (e.g., sense or antisense nucleic acid molecules encoding AK or AMPK or the polypeptides they encode) or can be identified, e.g., using the screening assays described herein (e.g., an AK or AMPK agonist or antagonist, a peptidomimetic of an AK or AMPK agonist or antagonist, a peptidomimetic, or other small molecule).

Modulatory methods of the invention involve contacting a cell (e.g., an adipocyte) with an agent that modulates the activity and/or expression of AK or AMPK molecule. In certain embodiments the agent used inhibits the activity and/or expression AK (e.g AK1 or AK3).

These modulatory methods can be performed in vitro (e.g., by contacting the cell with the agent) or, alternatively, in vivo (e.g., by administering the agent to a subject). As such, the present invention provides methods of treating an individual afflicted with a condition or disorder that would benefit from modulation of the balance between energy consumption and energy generation. In one embodiment, the method involves administering an agent (e.g., an agent identified by a screening assay described herein), or combination of agents that modulates (e.g., upregulates or downregulates) AK or AMPK expression and/or activity. In particular embodiments of the agent inhibits the activity and/or expression of AK (e.g. AK1 or AK3).

Exemplary agents for use in upmodulating AK or AMPK activity include, e.g., nucleic acid molecules encoding AK or AMPK polypeptides, and compounds that stimulate the expression and/or activity of AK or AMPK.

Exemplary agents for use in downmodulating AK or AMPK activity include (e.g., substrate analogs that are not catalytically acted upon or that are not biologically active (e.g., nucleotide analogs), antisense molecules, siRNA molecules, dominant negative mutants, or compounds identified in the subject screening assays).

i. Exemplary Inhibitory Compounds

Inhibitory compounds of the invention can be, for example, intracellular binding molecules that act to specifically inhibit the expression or activity of AK or AMPK. As used herein, the term “intracellular binding molecule” is intended to include molecules that act intracellularly to inhibit the expression or activity of a protein by binding to the protein or to a nucleic acid (e.g., an mRNA molecule) that encodes the protein. Examples of intracellular binding molecules, described in further detail below, include antisense nucleic acids, intracellular antibodies, peptidic compounds that inhibit the interaction of AK or AMPK with a target molecule (e.g., a substrate) and chemical agents that specifically inhibit AK or AMPK activity. In certain embodiments of the invention exemplary inhibitory compounds inhibit the expression or activity of AK. In certain preferred embodiments the compound specifically inhibits AK1 and/or AK3.

a. Antisense Nucleic Acid Molecules

In one embodiment, an inhibitory compound of the invention is an antisense nucleic acid molecule that is complementary to a gene encoding AK (e.g. AK1 or AK3) or AMPK or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the coding strand sequences encoding AK or AMPK disclosed herein, antisense nucleic acids of the invention can be designed according to the rules of Watson and Crick base pairing. The antisense nucleic acid molecule can be complementary to the entire coding region of AK or AMPK mRNA, but more preferably is an oligonucleotide which is antisense to only a portion of the coding or noncoding region of AK or AMPK mRNA. For example, the antisense oligonucleotide can be complementary to the region surrounding the translation start site of AK or AMPK mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides may be used. Examples of modified nucleotides which may be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a AK or AMPK to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330). In another embodiment, an antisense nucleic acid of the invention is a compound that mediates RNAi. RNA interfering agents include, but are not limited to, nucleic acid molecules including RNA molecules which are homologous to the target gene or genomic sequence, e.g., AK or AMPK, or a fragment thereof, “short interfering RNA” (siRNA), “short hairpin” or “small hairpin RNA” (shRNA), and small molecules which interfere with or inhibit expression of a target gene by RNA inerference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591) may be used to catalytically cleave AK or AMPK mRNA transcripts to thereby inhibit translation of AK or AMPK mRNA.

Alternatively, gene expression can be inhibited by targeting nucleotide sequences complementary to the regulatory region of AK or AMPK to form triple helical structures that prevent transcription of the AK or AMPK gene in target cells. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12):807-15.

In yet another embodiment, the AK or AMPK nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al., 1996, Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al., 1996, supra; Perry-O'Keefe et al., 1996, Proc. Natl. Acad. Sci. USA 93: 14670-675.

PNAs of AK or AMPK nucleic acid molecules may be used in therapeutic and diagnostic applications. For example, PNAs may be used as antisense or antigene agents for sequence-specific modulation of gene expression by, for example, inducing transcription or translation arrest or inhibiting replication. PNAs of AK or AMPK nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B., 1996, supra)); or as probes or primers for DNA sequencing or hybridization (Hyrup B. et al., 1996, supra; Perry-O'Keefe supra).

In another embodiment, PNAs of AK or AMPK can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of AK or AMPK nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B., 1996, supra). The synthesis of PNA-DNA chimeras can be performed as described in Hyrup B., 1996, supra and Finn P. J. et al., 1996, Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, may be used as a between the PNA and the 5′ end of DNA (Mag, M. et al., 1989, Nucleic Acid Res. 17: 5973-88). PNA monomers are then coupled in a stepwise manner to produce a chimeric molecule with a 5′ PNA segment and a 3′ DNA segment (Finn P. J. et al., 1996, supra). Alternatively, chimeric molecules can be synthesized with a 5′ DNA segment and a 3′ PNA segment (Peterser, K. H. et al., 1975, Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. US. 86:6553-6556; Lemaitre et al., 1987, Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al., 1988, Bio-Techniques 6:958-976) or intercalating agents. (See, e.g., Zon, 1988, Pharm. Res. 5:539-549). To this end, the oligonucleotide may be conjugated to another molecule, (e.g., a peptide, hybridization triggered cross-linking agent, transport agent, or hybridization-triggered cleavage agent).

Antisense polynucleotides may be produced from a heterologous expression cassette in a transfectant cell or transgenic cell. Alternatively, the antisense polynucleotides may comprise soluble oligonucleotides that are administered to the external milieu, either in the culture medium in vitro or in the circulatory system or in interstitial fluid in vivo. Soluble antisense polynucleotides present in the external milieu have been shown to gain access to the cytoplasm and inhibit translation of specific mRNA species.

b. Intracellular Antibodies

Another type of inhibitory compound that may be used to inhibit the expression and/or activity of AK or AMPK in a cell is an intracellular antibody specific for AK or AMPK discussed herein. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA.91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of transcription factor activity according to the inhibitory methods of the invention, preferably an intracellular antibody that specifically binds the transcription factor is expressed within the nucleus of the cell. Nuclear expression of an intracellular antibody can be accomplished by removing from the antibody light and heavy chain genes those nucleotide sequences that encode the N-terminal hydrophobic leader sequences and adding nucleotide sequences encoding a nuclear localization signal at either the N— or C-terminus of the light and heavy chain genes (see e.g., Biocca, S. et al. (1990) EMBO J. 9:101-108; Mhashilkar, A. M. et al. (1995) EMBO J. 14:1542-1551). A preferred nuclear localization signal to be used for nuclear targeting of the intracellular antibody chains is the nuclear localization signal of SV40 Large T antigen (see Biocca, S. et al. (1990) EMBO J. 9:101-108; Mhashilkar, A. M. et al. (1995) EMBO J. 14:1542-1551).

To prepare an intracellular antibody expression vector, antibody light and heavy chain cDNAs encoding antibody chains specific for the target protein of interest, e.g., AK or AMPK protein, is isolated, typically from a hybridoma that secretes a monoclonal antibody specific for AK or AMPK protein. For example, antibodies can be prepared by immunizing a suitable subject, (e.g., rabbit, goat, mouse or other mammal) with a AK or AMPK protein immunogen. An appropriate immunogenic preparation can contain, for example, recombinantly expressed AK or AMPK protein or a chemically synthesized AK or AMPK peptide. The preparation can further include an adjuvant, such as Freund's complete or incomplete adjuvant, or similar immunostimulatory compound. Antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a AK or AMPK immunogen as described above, and the culture supernatants of the resulting hybridoma cells are screened to identify a hybridoma producing a monoclonal antibody that binds specifically to the AK or AMPK protein. Any of the many well known protocols used for fusing lymphocytes and immortalized cell lines can be applied for the purpose of generating an anti-AK or AMPK protein monoclonal antibody (see, e.g., G. Galfre et al. (1977) Nature 266:550-52; Gefter et al. Somatic Cell Genet., cited supra; Lemer, Yale J. Biol. Med., cited supra; Kenneth, Monoclonal Antibodies, cited supra). Moreover, the ordinary skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection.(ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days -because they are not transformed). Hybridoma cells producing a monoclonal antibody that specifically binds the maf protein are identified by screening the hybridoma culture supernatants for such antibodies, e.g., using a standard ELISA assay.

Alternative to preparing monoclonal antibody-secreting hybridomas, a monoclonal antibody that binds to a AK or AMPK can be identified and isolated by screening a recombinant combinatorial immunoglobulin library (e.g., an antibody phage display library) with the protein, or a peptide thereof, to thereby isolate immunoglobulin library members that bind specifically to the protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and compounds particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Once a monoclonal antibody of interest specific for AK or AMPK has been identified (e.g., either a hybridoma-derived monoclonal antibody or a recombinant antibody from a combinatorial library, including monoclonal antibodies to AK or AMPK that are already known in the art), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. As discussed above, the sequences encoding the hydrophobic leaders of the light and heavy chains are removed and sequences encoding a nuclear localization signal (e.g., from SV40 Large T antigen) are linked in-frame to sequences encoding either the amino- or carboxy terminus of both the light and heavy chains. The expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit transcription factor activity in a cell, the expression vector encoding the AK or AMPK specific intracellular antibody is introduced into the cell by standard transfection methods.

c. Peptidic Compounds

In another embodiment, an inhibitory compound of the invention is a peptidic compound derived from the AK (e.g AK1 or AK3)or AMPK amino acid sequence. In particular, the inhibitory compound comprises a portion of AK or AMPK (or a mimetic thereof) that mediates interaction of AK or AMPK with a target molecule such that contact of AK or AMPK with this peptidic compound competitively inhibits the interaction of AK or AMPK with the target molecule.

The peptidic compounds of the invention can be made intracellularly in cells by introducing into the cells an expression vector encoding the peptide. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like).

Other inhibitory agents that may be used to specifically inhibit the activity of an AK or AMPK protein are chemical compounds that directly inhibit AK or AMPK activity or inhibit the interaction between AK or AMPK molecule and target molecules. Such compounds can be identified using screening assays that select for such compounds, as described in detail above.

ii. Exemplary Stimulatory Compounds

In the stimulatory methods of the invention, a subject is treated with a stimulatory compound that stimulates expression and/or activity of a AK or AMPK.

Examples of stimulatory compounds include active AK or AMPK protein, expression vectors encoding AK or AMPK and chemical agents that specifically stimulate AK or AMPK activity.

A preferred stimulatory compound is a nucleic acid molecule encoding AK or AMPK molecule, wherein the nucleic acid molecule is introduced into the subject (e.g., adipocytes of the subject) in a form suitable for expression of the AK or AMPK protein in the cells of the subject. For example, a AK or AMPK cDNA (full length or partial. AK or AMPK cDNA sequence) is cloned into a recombinant expression vector and the vector is transfected into the immune cell using standard molecular biology techniques. The AK or AMPK cDNA can be obtained, for example, by amplification using the polymerase chain reaction (PCR) or by screening an appropriate cDNA library. The, nucleotide sequences of AK or AMPK cDNA is known in the art and may be used for the design of PCR primers that allow for amplification of a cDNA by standard PCR methods or for the design of a hybridization probe that may be used to screen a cDNA library using standard hybridization methods.

Following isolation or amplification of AK or AMPK cDNA, the DNA fragment is introduced into a suitable expression vector, as described above. Nucleic acid molecules encoding AK or AMPK in the form suitable for expression in a host cell, can be prepared as described above using nucleotide sequences known in the art. The nucleotide sequences may be used for the design of PCR primers that allow for amplification of a cDNA by standard PCR methods or for the design of a hybridization probe that may be used to screen a cDNA library using standard hybridization methods.

Another form of a stimulatory compound for stimulating expression of AK or AMPK in a cell is a chemical compound that specifically stimulates the expression or activity of endogenous AK or AMPK in the cell. Such compounds can be identified using screening assays that select for compounds that stimulate the expression or activity of AK or AMPK as described herein.

The method of the invention for modulating AK or AMPK activity in a subject can be practiced either in vitro or in vivo (the latter is discussed further in the following subsection). For practicing the method in vitro, cells (e.g., adipocytes) can be obtained from a subject by standard methods and incubated (i.e., cultured) in vitro with a stimulatory or inhibitory compound of the invention to stimulate or inhibit, respectively, the activity of AK or AMPK. Cells treated in vitro with either a stimulatory or inhibitory compound can be administered to a subject to influence the growth and/or differentiation of cells in the subject. For further discussion of ex vivo genetic modification of cells followed by readministration to a subject, see also U.S. Pat. No. 5,399,346 by W. F. Anderson et al.

In other embodiments, a stimulatory or inhibitory compound is administered to a subject in vivo, such as directly to an articulation site of a subject. For stimulatory or inhibitory agents that comprise nucleic acids (e.g., recombinant expression vectors encoding AK or AMPK molecule, antisense RNA, intracellular antibodies or AK or AMPK-derived peptides), the compounds can be introduced into cells of a subject using methods known in the-art for introducing nucleic acid (e.g., DNA) into cells in vivo. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo may be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm may be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which may be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes; bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018 Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and may be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 may be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

VI. Diagnostic Assays

In another aspect, the invention features a method of diagnosing a subject for a disorder that would benefit from modulation of the activity of one or more of: AK or AMPK. Exemplary disorders include those that would benefit from modulation of the, balance between energy generation and energy consumption.

In one embodiment, the expression of AK or AMPK or a molecule in cells of a subject may be measured and compared to a control and a difference in expression of AK or AMPK in cells of the subject as compared to the control could be used to diagnose the subject as one that would benefit from modulation of AK or AMPK activity.

The “change in expression” or “difference in expression” may be detected by assaying levels of mRNA, for example, by isolating cells from the subject and determining the level of mRNA expression in the cells by standard methods known in the art, including Northern blot analysis, microarray analysis, reverse-transcriptase PCR analysis and in situ hybridizations. For example, a biological specimen can be obtained from the patient and assayed for, e.g., expression or activity of AK or AMPK.

In another embodiment, protein expression may be measured using standard methods known in the art, including Western blot analysis, immunoprecipitations, enzyme linked immunosorbent assays (ELISAs) and immunofluorescence. Antibodies for use in such assays can be made using techniques known in the art and/or as described herein for making intracellular antibodies.

In another embodiment, a change in expression of AK or AMPK in cells of the subject results from one or more mutations (i.e., alterations from wild-type), e.g., the AK or AMPK gene and mRNA leading to one or more mutations (i.e., alterations from wild-type) in the amino acid sequence of the protein. In one embodiment, the mutation(s) leads to a form of the molecule with increased activity (e.g., partial or complete constitutive activity). In another embodiment, the mutation(s) leads to a form of the molecule with decreased activity (e.g., partial or complete inactivity). The mutation(s) may change the level of expression of the molecule for example, increasing or decreasing the level of expression of the molecule in a subject with a disorder. Mutations in the nucleotide sequence or amino acid sequences of proteins can be determined using standard techniques for analysis of DNA or protein sequences, for example for DNA or protein sequencing, RFLP analysis, and analysis of single nucleotide or amino acid polymorphisms. For example, in one embodiment, mutations can be detected using highly sensitive PCR approaches using specific primers flanking the nucleic acid sequence of interest. In one embodiment, detection of the. alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364). This method can include the steps of collecting a sample of cells from a patient, isolating nucleic acid (e.g., genomic, DNA) from the cells of the sample, contacting the nucleic acid sample with one or more primers which specifically amplify a sequence under conditions such that hybridization and amplification of the sequence (if present) occurs, and detecting the presence or absence of an amplification product, or detecting the size of the amplification product and comparing the length to a control sample.

In one embodiment, the complete nucleotide sequence for AK or AMPK can be determined. Particular techniques have been developed for determining actual sequences in order to study polymorphism in human genes. See, for example, Proc. Natl. Acad. Sci. U.S.A. 85, 544-548 (1988) and Nature 330, 384-386 (1987); Maxim and Gilbert. 1977. PNAS 74:560; Sanger 1977. PNAS 74:5463. In addition, any of a variety of automated sequencing procedures can be utilized when performing diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Restriction fragment length polymorphism mappings (RFLPS) are based on changes at a restriction enzyme site. In one embodiment, polymorphisms from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Moreover, the use of sequence specific ribozymes (see, for example, U.S. Pat. No. 5,498,531) may be used to score for the presence of a specific ribozyme cleavage site.

Another technique for detecting specific-polymorphisms in particular DNA segment involves hybridizing DNA segments which are being analyzed (target DNA) with a complimentary, labeled oligonucleotide probe. See Nucl. Acids Res. 9, 879-894 (1981). Since DNA duplexes containing even a single base pair mismatch exhibit high thermal instability, the differential melting temperature may be used to distinguish target DNAs that are perfectly complimentary to the probe from target DNAs that only differ by a single nucleotide. This method has been adapted to detect the presence or absence of a specific restriction site, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for detecting polymorphisms in nucleic acid sequences include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the polymorphic sequence with potentially polymorphic RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In another embodiment, alterations in electrophoretic mobility may be used to identify polymorphisms. For example, single strand conformation polymorphism (SSCP) may be used to detect differences in electrophoretic mobility between mutant and wild-type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control nucleic acids can be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of nucleic acid molecule comprising polymorphic sequences in polyacrylamide gels containing a gradient of denaturant is assayed using denaturing gradient gel electrophoresis (DGGE) (Myers et al. (1985) Nature 313:495). When DGGE is used as the method of analysis, DNA can be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting polymorphisms include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different polymorphisms when the oligonucleotides are attached to the hybridizing membrane and hybridized with labeled target DNA.

Another process for studying differences in DNA structure is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product.

Another process exploits the fact that the incorporation of some nucleotide analogs into DNA causes an incremental shift of mobility when the DNA is subjected to a size fractionation process, such as electrophoresis. Nucleotide analogs may be used to identify changes since they can cause an electrophoretic mobility shift. See, U.S. Pat. No. 4,879,214.

Many other techniques for identifying and detecting polymorphisms are known to those skilled in the art, including those described in “DNA Markers: Protocols, Applications and Overview,” G. Caetano-Anolles and P. Gresshoff ed., (Wiley-VCH, New York) 1997, which is incorporated herein by reference as if fully set forth.

In addition, many approaches have also been used to specifically detect SNPs. Such techniques are known in the art and many are described e.g., in DNA Markers: Protocols, Applications, and Overviews. 1997. Caetano-Anolles and Gresshoff, Eds. Wiley-VCH, New York, pp 199-21 1 and the references contained therein). For example, in one embodiment, a solid phase approach to detecting polymorphisms such as SNPs may be used. For example an oligonucleotide ligation assay (OLA) may be used. This assay is based on the ability of DNA ligase to distinguish single nucleotide differences at positions complementary to the termini of co-terminal probing oligonucleotides (see, e.g., Nickerson et al. 1990. Proc. Natl. Acad. Sci. USA 87:8923. A modification of this approach, termed coupled amplification and oligonucleotide ligation (CAL) analysis, has been used for multiplexed genetic typing (see, e.g., Eggerding 1995 PCR Methods Appl. 4:337); Eggerding et al. 1995 Hum. Mutat. 5:153).

In another embodiment, genetic bit analysis (GBA) may be used to detect a SNP (see, e.g., Nikiforov et al. 1994. Nucleic Acids Res. 22:4167; Nikiforov et al. 1994. PCR Methods Appl. 3:285; Nikiforov et al. 1995. Anal Biochem. 227:201). In another embodiment, microchip electrophoresis may be used for high-speed SNP detection (see e.g., Schmalzing et al. 2000. Nucleic Acids Research, 28). In another embodiment, matrix-assisted laser desorption/ionization time-of-flight mass (MALDI TOF) mass spectrometry may be used to detect SNPs (see, e.g., Stoerker et al. Nature Biotechnology 18:1213).

In another embodiment, a difference in a biological activity of AK or AMPK between a subject and a control can be detected.

In preferred embodiments, the diagnostic assay is conducted on a biological sample from the subject, such as a cell sample or a tissue section (for example, a freeze-dried or fresh frozen section of tissue removed from a subject). In another embodiment, the level of expression of AK or AMPK in cells of the subject can be detected in vivo, using an appropriate imaging method, such as using a radiolabeled antibody.

In certain embodiments, the level of expression of AK or AMPK in cells of the test subject may be elevated (i.e., increased) relative to the control not associated with the disorder or the subject may express a constitutively active (partially or completely) form of the molecule. This elevated expression level of, e.g., AK or AMPK or expression of a constitutively active form of AK or AMPK, may be used to diagnose a subject for a disorder associated with increased AK or AMPK activity. For example, in certain preferred embodiments, the diagnostic methods of the invention are used to detect elevated levels of activity of AK (e.g. AK1 or AK3) relative to control subjects to identify subjects in need of treatment with an AK1 inhibitor, e.g., in disorders related to insufficient energy consumption such as obesity and diabetes.

In another embodiment, the level of expression of AK or AMPK in cells of the subject may be reduced (i.e., decreased) relative to the control not associated with the disorder or the subject may express an inactive (partially or completely) mutant form of AK or AMPK. This reduced expression level of AK or AMPK or expression of an inactive mutant form of AK or AMPK may be used to diagnose a subject for a disorder.

The methods described herein maybe performed, for example, by utilizing pre-packaged diagnostic kits comprising at least one probe/primer nucleic acid or other reagent (e.g., antibody), which may be conveniently used, e.g., in clinical settings to diagnose patients exhibiting symptoms or family history of a disease or illness involving an energy related state (e.g, involving AK or AMPK).

VII. Kits of the Invention

Another aspect of the invention pertains to kits for carrying out the screening assays, modulatory methods or diagnostic assays of the invention. For example, a kit for carrying out a screening assay of the invention may include an indicator composition comprising AK or AMPK, means for measuring a readout (e.g., protein secretion) and instructions for using the kit to identify modulators of biological effects of AK or AMPK. In another embodiment, a kit for carrying out a screening assay of the invention may include cells deficient in AK or AMPK, means for measuring the readout and instructions for using the kit to identify modulators of a biological effect of AK or AMPK, e.g., the balance between energy generation and energy consumption.

In another embodiment, the invention provides a kit for carrying out a modulatory method of the invention. The kit can include, for example, a modulatory agent of the invention (e.g., AK or AMPK inhibitory or stimulatory agent) in a suitable carrier and packaged in a suitable container with instructions for use of the modulator to modulate a biological effect of AK or AMPK, e.g., the balance between energy generation and consumption.

Another aspect of the invention pertains to a kit for diagnosing a disorder associated with AK or AMPK expression and/or activity in a subject. The kit can include a reagent for determining expression of AK or AMPK (e.g., a nucleic acid probe for detecting AK or AMPK mRNA or an antibody for detection of AK or AMPK), a control to which the results of the subject are compared, and instructions for using the kit for diagnostic purposes.

VIII. Administration of Modulating Agents

Modulatory agents (e.g. AK inhibitors, e.g. AK1 or AK3)of the invention are administered to subjects in a biologically compatible form suitable for pharmaceutical administration in vivo to either enhance or suppress immune responses (e.g., T cell mediated immune responses). By “biologically compatible form suitable for administration in vivo” is meant a form of the protein to be administered in which any toxic effects are outweighed by the therapeutic effects of the modulating agent. The term subject is intended to include living organisms in which an immune response can be elicited, e.g., mammals. Examples of subjects include humans, dogs, cats, mice, rats, and transgenic species thereof, including but not limited to the transgenic AK or AMPK mouse described herein. Administration of an agent as described herein can be in any pharmacological form including a therapeutically active amount of an agent alone or in combination with a pharmaceutically acceptable carrier.

Administration of a therapeutically active amount of the therapeutic compositions of the present invention is defined as an amount effective, at dosages and for periods of time necessary to achieve the desired result. For example, a therapeutically active amount of a modulating agent may vary according to factors such as the disease state, age, sex, and weight of the individual, and the ability of peptide to elicit a desired response in the individual. Dosage regimen may be adjusted to provide the optimum therapeutic response. For example, several divided doses may be administered daily or the dose may be proportionally reduced as indicated by the exigencies of the therapeutic situation.

The therapeutic or pharmaceutical compositions of the present invention can be administered by any suitable route known in the art including for example intravenous, subcutaneous, intramuscular, transdermal, intrathecal or intracerebral or administration to cells in ex vivo treatment protocols. Administration can be either rapid as by injection or over a period of time as by slow infusion or administration of slow release formulation. For treating tissues in the central nervous system, administration can be by injection or infusion into the cerebrospinal fluid (CSF). When it is intended that a modulator be administered to cells in the central nervous system, administration can be with one or more agents capable of promoting penetration of modulator across the blood-brain barrier.

The modulator can also be linked or conjugated with agents that provide desirable pharmaceutical or pharmacodynamic properties. For example, modulator can be coupled to any substance known in the art to promote penetration or transport across the blood-brain barrier such as an antibody to the transferrin receptor, and administered by intravenous injection. (See for example, Friden et al., 1993, Science 259: 373-377 which is incorporated by reference). Furthermore, modulator can be stably linked to a polymer such as polyethylene glycol to obtain desirable properties of solubility, stability, half-life and other pharmaceutically advantageous properties. (See for example Davis et al., 1978, Enzyme Eng 4: 169-73; Burnham, 1994, Am J Hosp Pharm 51: 210-218, which are incorporated by reference).

Furthermore, the modulator can be in a composition which aids in delivery into the cytosol of a cell. For example, the agent may be conjugated with a carrier moiety such as a liposome that is capable of delivering the peptide into the cytosol of a cell. Such methods are well known in the art (for example see Amselem et al., 1993, Chem Phys Lipids 64: 219-237, which is incorporated by reference). Alternatively, the AK or AMPK modulator can be modified to include specific transit peptides or fused to such transit peptides which are capable of delivering the modulator into a cell. In addition, the agent can be delivered directly into a cell by microinjection.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier”[includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. AK or AMPK can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Dose administration can be repeated depending upon the pharmacokinetic parameters of the dosage formulation and the route of administration used. It is also provided that certain formulations containing the modulator are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, olyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies may be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method for the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

In one embodiment of this invention, a modulatory agent may be therapeutically administered by implanting into patients vectors or cells capable of producing, for example a biologically-active form of AK or AMPK or a precursor of AK or AMPK, i.e. a molecule that can be readily converted to a biological-active form AK or AMPK by the body. In one approach cells that secrete AK or AMPK may be encapsulated into semipermeable membranes for implantation into a patient. The cells can be cells that normally express AK or AMPK or a precursor thereof or the cells can be transformed to AK or AMPK or a biologically active fragment thereof or a precursor thereof. It is preferred that the cell be of human origin and that the AK or AMPK polypeptide be human AK or AMPK when the patient is human. However, the formulations and methods herein may be used for veterinary as well as human applications and the term “patient” or “subject” as used herein is intended to include human and veterinary patients.

Monitoring the influence of agents (e.g., drugs or compounds) on the expression or activity an AK or AMPK gene can be applied not only in basic drug screening, but also in clinical trials. For example, the effectiveness of an agent determined by a screening assay as described herein to increase AK or AMPK gene expression, protein levels, or upregulate enzymatic activity, can be monitored in clinical trials of subjects exhibiting decreased AK or AMPK gene expression, protein levels, or downregulated AK or AMPK activity. Alternatively, the effectiveness of an agent determined by a screening assay to decrease AK or AMPK gene expression, protein levels, or downregulateenzymatic activity, can be monitored in clinical trials of subjects exhibiting increased AK or AMPKe gene expression, protein levels, or upregulated cytokine activity. In such clinical trials, the expression or activity of an AK or AMPK gene, and preferably, other genes that have been implicated in a disorder may be used as a “read out” or markers of the phenotype of a particular cell.

For example, and not by way of limitation, genes, including AK or AMPK, that are modulated in cells by treatment with an agent (e.g., compound, drug or small molecule) which modulates AK or AMPK activity (e.g., identified in a screening assay as described herein) can be identified. Thus, to study the effect of agents on a AK or AMPK associated disorder, for example, in a clinical trial, cells can be isolated and RNA prepared and analyzed for the levels of expression of AK or AMPK and other genes implicated in the disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of AK or AMPK or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a AK and/or AMPKprotein, mRNA, or genomic DNA in the pre-administration sample; (iii) obtaining one or more post-administration samples from the subject; (iv) detecting the level of expression or activity of the AK or AMPK protein, mRNA, or genomic DNA in the post-administration samples; (v) comparing the level of expression or activity of the AK or AMPK protein, mRNA, or genomic DNA in the pre-administration sample with the AK or AMPK protein, mRNA, or genomic DNA in the post administration sample or samples; and (vi) altering the administration of the agent to the subject accordingly. For example, increased administration of the agent may be desirable to increase the expression or activity of AK or AMPK to higher levels than detected, i.e., to increase the effectiveness of the agent. Alternatively, decreased administration of the agent may be desirable to decrease expression or activity of AK or AMPK to lower levels than detected, i.e. to decrease the effectiveness of the agent. According to such an embodiment, AK or AMPK expression or activity may be used as an indicator of the effectiveness of an agent, even in the absence of an observable phenotypic response.

In a preferred embodiment, the ability of an AK or AMPK modulating agent to modulate AK (e.g AK1 or AK3) or AMPK in a cell (e.g. an adipocyte cell or muscle cell) of a subject that would benefit from modulation of the expression and/or activity of a cytokine gene can be measured by detecting an improvement in the condition of the patient after the administration of the agent. Such improvement can be readily measured by one of ordinary skill in the art using indicators appropriate for the specific condition of the patient (e.g. weight, glucose levels, muscle density, fat accumulation). Monitoring the response of the patient by measuring changes in the condition of the patient is preferred in situations where the collection of biopsy materials would pose an increased risk and/or detriment to the patient.

The present invention also provides methods for detecting the presence of AK or AMPK a sample from a patient.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

This invention is further illustrated by the following examples which should not be construed as limiting. The contents of all references, patents and published patent applications cited throughout this application are hereby incorporated by reference. Nucleotide and amino acid sequences deposited in public databases as referred to herein are also hereby incorporated by reference.

### EXEMPLIFICATION

The following materials, methods, and examples are illustrative and not intended to be limiting.

### Example I

### Stress-Induced Expression of Adenylate Kinase by Tunicamycin Treatment on Adipocyte cells

To determine what proteins were upregulated when cells were exposed to stress, differentiated 3T3-L1 adipocytes were treated with Tunicamycin (an inhibitor of N-linked glycoprotein synthesis) at a final concentration of 5 μg/mL for 5 hours. After 5 hours, adipocytes cells were additionally treated with ³⁵S-methionine for 30 minutes. Adipocytes were washed two times with ice-cold phosphate buffered-solution (PBS) and washed one time with isolation buffer (250 mM sucrose, 0.5 mM EGTA, and 5 mM Hepes, pH 7.4). Adipocytes were homogenized by seven passages through a 27-g needle and centrifuiged at 500×g for 10 minutes. The post-nuclear supernatant (PNS) was removed and saved. Isolation buffer was added to the post-nuclear pellet, the pellet was homogenized as indicated above and the homogenized suspension was centrifuged for 3 min at 500×g. The PNS was removed, combined with the first PNS and centrifuged at 18,000×g for 25 minutes. The pellet was resuspended in 20% sucrose, 10 mM Tris and 0.1 mM EDTA and centrifuged at 18,000×g for 30 minutes. The pellet was resuspended in 60% sucrose, 10 mM Tris and 0.05 mM EDTA. The suspension was overlayed with a 53% sucrose layer (sucrose, 10 mM Tris, and 0.05 mM EDTA) and a 44% sucrose layer (sucrose, 10 mM Tris and 0.05 mM EDTA). The sucrose step gradient was centrifuged at 141,000×g for 2 hours. The purified mitochondria settled at the 44/53 interface. The mitochondrial layer was removed, diluted in isolation buffer and centrifuiged at 18,000×g for 30 min. The mitochondrial pellet was suspended in 50 μL of isolation buffer and a protein determination assay performed by the Bradford assay (Bio-Rad). CHAPS detergent was added to the suspension to a final concentration of 6 mM CHAPS, the suspension was vortexed, and isolation buffer then added to bring the final concentration of CHAPS to 3 mM. The suspension was vortexed continuously for 10 min. Equal concentrations of protein were layered on top of a 1.8 ml 10%-30% sucrose gradient, and spun at 35,000 rpm in a SW 50.1 rotor (Beckman) at 4° C. for 20 h. The gradient was fractionated into 10×200 μL fractions, which were then analyzed by SDS-PAGE. Gels were then analyzed either by SyproRuby protein staining or by phosphorimaging following transfer to a nitrocellulose membrane. The purification scheme determined a single band from the cell fractionation that was associated with mitochondrial DNA. Mass spectrometry was used to identify the unknown band as adenylate kinase 3 (AK3).

### Example II

### Specific Silencing of AK Expression by SiRNA in Adipocyte Cells

In order to determine the importance of AK in the maintenance of AMP to ATP ratios and ultimately AMPK activation, the following experiment seeks to selectively inhibit the expression of AK protein kinases in intact cultured adipocytes through the use of interference RNA. First discovered in Caenorhabditis elegans (Fire et al. (1998) Nature 391:806-811), this gene-silencing technique uses double-stranded RNA to activate nuclease-containing protein complexes (RNA-induced silencing complex) to target a specific mRNA species, which is then degraded (Hammond et al. (2001) Nature 404:293-296; Hammond et al. (2001) Science 293:1146-1150). Before insertion into protein-silencing complexes, processing of double-stranded RNA into small interfering RNA (siRNA) duplexes of 21-23 nt occurs by enzymes known as Dicers (Bernstein et al. (2001) Nature 409:363-366; Zamore et al. (2000) Cell 101:25-33; Elbashir et al. (2000) Genes Dev. 15:188-200). Extension of the technique to mammalian cells has involved the use of synthetic siRNA duplexes of 21-base lengths transfected directly into cultured cells, where decreased levels of selected proteins can be observed in response to siRNA after 24-72 h (Elbashir et al. (2001) Nature 411:494-498).

Methods

3T3-L1 Adipocyte Differentiation:

3T3-L1 fibroblasts (American Type Culture Collection) are grown on 150 mm dishes in Dulbecco's Modified Eagle medium (DMEM) supplemented with 10% fetal bovine serum (FBS), 100 U/mL penicillin and 100 μg/mL streptomycin, and grown under 10% CO₂. 3T3-L1 fibroblasts 3 days postconfluent are differentiated into adipocytes by incubating with DMEM supplemented with the same antibiotics, 10% FBS, 0.5 mM 3-isobutyl-1-methylxanthine (Sigma), 0.25 μM dexamethasone (Sigma) and 1 μM insulin (Sigma) for 48 h. Cells are grown in DMEM with the same antibiotics and 10% FBS for an additional 5-6 days.

Electroporation of 3T3-L1 Adipocytes:

3T3-L1 adipocytes are transfected with siRNA complexes by electroporation. Adipocytes at day 5 of differentiation are detached from culture dishes with 0.25% trypsin and 0.5 mg of collagenase/ml in PBS, washed twice, and resuspended in PBS. Approximately 5 million cells (half of the cells from one p150 dish) are then mixed with siRNA duplexes, which are delivered to the cells by a pulse of electroporation with a Bio-Rad gene pulser II system at the setting of 0.18 kV and 960 μF capacitance. After electroporation, cells are immediately mixed with fresh medium for 10 min before reseeding onto culture dishes. The gradient is fractionated into 10×200 μL fractions, which are then analyzed by SDS-PAGE. Gels are then analyzed either by SyproRuby protein staining or by phosphorimaging following transfer to a nitrocellulose membrane.

Design and Synthesis of siRNA Duplexes:

The 21-mer sense and antisense strands of RNA oligonucleotides are designed as described in Elbashir et al. (2001) Nature 411:494-498. The AK 1, AK 2 and AK3 RNA oligonucleotides (TABLE 1) are synthesized in the 2′-ACE(TM) protected form, which enhances overall RNA stability and resistance to nucleases. The complementary sense and antisense strands of RNA oligonucleotides are mixed, 2′-deprotected, annealed, and purified by PAGE. Gel-purified duplexes are subsequently desalted by using reverse-phase column chromatography, followed by washing with 75% ethanol twice to ensure complete salt removal and dried by use of a Speed-Vac. The pellets are resuspended in nuclease-free water before transfection into cultured cells.

TABLE 1
AK 1, AK 2 and AK3 RNA Oligonucleotides

Adenylate Kinase 1:
GCTCCGAGATGCTATGTTA
GCGAGAAGATTGTACAGAA
GAAACAGGGAGAAGAATTT
GCTGGAGACTTATTACAAT
Adenylate Kinase 2:
GGTCCTACCATGAGGAATT
GGAAAAAAGCTGAAGGCGA
CGAAATGGTTGTGGAGCTA
CGACAATGGATGCAGGGAA
Adenylate Kinase 3:
GAACAGAAACCAACAAGAT
GAAAGCTGATCCCAGATGA
GTATACTCCTTCCTACAGA
GATAGACACAGTGATAAAT

Western Blotting:

Cells treated with siRNA are harvested as described above and lysed in ice-cold reporter lysis buffer (Promega) containing protease inhibitor (complete, EDTA-free, 1 tablet/10 ml buffer, Roche Molecular Biochemicals). After clearing the resulting lysates by centrifugation, protein in clear lysates are quantified by Dc protein assay kit (Bio-Rad). Proteins in 60 μg of total cell lysate are resolved by 10% SDS-PAGE, transferred onto a polyvinylidene difluoride membrane (PVDF, Bio-Rad), and immuno-blotted with antibodies against AK3, AK2 and AK3 (Santa Cruz). Protein content is visualized with a BM Chemiluminescence Blotting Kit (Roche Molecular Biochemicals). The blots are exposed to x-ray film (Kodak MR-1) for various times (30 s to 5 min).

### Example III

### Identification of Inhibitors that Modulates AK or AMPK Activity

In order to screen for inhibitors of AK or AMPK activity, 3T3-L1 adipocytes cels are transfected with a protein of interest i.e., AMPK and/or AK. Transfectants expressing the protein of interest are then isolated and contacted with a test compound, obtainable using any of the numerous approaches in combinatorial library methods. The ability of the test compound to modulate AK or AMPK activity is determined.

### Example IV

### Metabolic Phenotype in AK-1 Knockout Mice

AK-1 knockout mice (such as those described in Pucar et al (2000) J. Biol. Chem. 275: 41424-41429), consisting of animals having little or no adenylate kinase-1 enzymatic activity, were subjected to experimental observation in comparison to control mice. Measurements using techniques well known in the art were employed for determining relative body weight, fat (e.g. fat accumulation) and muscle mass. These parameters were used as a means for evaluating the metabolic phenotype of AK-1 knockout mice.

Comparison of control to AK-1 knockout mice indicated that mice lacking adenylate kinase-1 activity had a higher average body weight than control mice, however, this weight difference could not be attributed to fat accumulation in the knockout mice. Rather, the difference in weight between control and AK-1 knockout mice could be accounted for by increased levels of muscle weight in AK-1 deficient mice.

Experiments using established methods involving the administration of high fat diets demonstrated that further phenotypic differences existed between control and AK-1 knockout mice. Specifically, mice lacking adenylate kinase-1 activity were significantly less susceptible to weight gain in response to a high fat diet than their control counterparts. It was also observed that the AK-1 knockout mice were more resistant to the accumulation of fat. In addition the animals lacking adenylate kinase-1 activity demonstrated lower blood glucose levels that control animals fed a high fat diet.

These findings demonstrate that lowered AK activity leads to enhanced AMP kinase activity in whole animals and confirms that compounds inhibiting or ablating AK (e.g. AK-1) activity may be useful in treating energy related disorders such as obesity or diabetes.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a modulator of AMP-activated protein kinase (AMPK), comprising contacting a composition comprising adenylate kinase (AK) or a bioactive fragment thereof and an adenylate kinase (AK) substrate or a bioactive fragment thereof with a test compound and determining the ability of the test compound to modulate interaction of AK or the AK bioactive fragment with the substrate or the substrate bioactive fragment, to thereby identify an AMPK modulator.

2. A method for identifying a modulator of AMPK, comprising contacting a composition comprising AK or a bioactive fragment thereof and an AK substrate or a bioactive fragment thereof with a test compound and determining the ability of the test compound to modulate an activity of AK or the AK bioactive fragment, to thereby identify an AMPK modulator.

3. A method for identifying a modulator of AMPK, comprising contacting a composition comprising AK or a bioactive fragment thereof and an AK substrate or a bioactive fragment thereof with a test compound and determining the ability of the test compound to modulate an activity of the substrate or the substrate bioactive fragment, to thereby identify an AMPK modulator.

4. A method for identifying a modulator of AMPK, comprising contacting a composition comprising AK or a bioactive fragment thereof and an AK substrate or a bioactive fragment thereof with a test compound and determining the ability of the test compound to modulate the phosphorylation state of the AK substrate or the substrate bioactive fragment, to thereby identify an AMPK modulator.

5. The method of claim 1, wherein the interaction of AK or the AK bioactive fragment with the substrate or the substrate bioactive fragment comprises binding of AK or the AK bioactive fragment to the substrate or the substrate bioactive fragment.

6. The method of any one of claims 1-4, wherein the AK substrate is AMP or ATP.

7. The method of any one of claims 1-4, wherein the AK is selected from the group consisting of AK1, AK2A, AK2B, AK2C, AK3, AK4 and AK5.

8. The method of any one of claims 1-4, wherein the AK is AK3 or AK1.

9. The method of any one of the claims 1-4, wherein the compound affects the interaction of AK with AMP, ATP or AMP and ATP.

10. The method of claim 9, wherein the compound affects the binding of AK with AMP, ATP or AMP and ATP.

11. The method of any one of the claims 1-4, wherein the compound affects the activity of AK on AMP, ATP or AMP and ATP.

12. The method of any one of claims 1-4, wherein at least one of AK, the AK bioactive fragment, the AK substrate or the substrate bioactive fragment is detectably labeled.

13. The method of any one of claims 12, wherein at least one of AK, the AK bioactive fragment, the AK substrate or the substrate bioactive fragment is radioactively labeled.

14. The method of any one of claims 12, wherein at least one of AK, the AK bioactive fragment, the AK substrate or the substrate bioactive fragment is fluorescently labeled.

15. The method of claim 1, wherein the interaction in the presence of the test compound is compared to interaction in the absence of the test compound.

16. The method of any one of claims 1-4, wherein at least one of AK, the AK bioactive fragment, the AK substrate or the substrate bioactive fragment is immobilized.

17. The method of claim 2, wherein the activity of AK or the bioactive fragment thereof regulates AMP to ATP ratios or regulates AMPK activation.

18. A method for identifying a modulator of AMPK, comprising contacting a cell that expresses an AK substrate or a bioactive fragment thereof and AK or a bioactive fragment thereof with a test compound and determining the ability of the test compound to modulate interaction of the AK substrate or the substrate bioactive fragment with AK or the AK bioactive fragment, to thereby identify an AMPK modulator.

19. A method for identifying a modulator of AMPK, comprising contacting a cell that expresses an AK substrate or a bioactive fragment thereof and AK or a bioactive fragment thereof with a test compound and determining the ability of the test compound to modulate an activity of the AK or the AK bioactive fragment, to thereby identify an AMPK modulator.

20. A method for identifying a modulator of AMPK, comprising contacting a cell that expresses an AK substrate or a bioactive fragment thereof and AK or a bioactive fragment thereof with a test compound and determining the ability of the test compound to modulate an activity of the AK substrate or the substrate bioactive fragment, to thereby identify an AMPK modulator.

21. A method for identifying an a modulator of AMPK, comprising contacting a cell that expresses AK or a bioactive fragment thereof and an AK substrate or a bioactive fragment thereof with a test compound and determining the ability of the test compound to modulate the phosphorylation state of the AK substrate or the substrate bioactive fragment, to thereby identify an AMPK modulator.

22. The method of claim 21, wherein the interaction of AK or the AK bioactive fragment with the substrate or the substrate bioactive fragment comprises binding of AK or the AK bioactive fragment to the substrate or the substrate bioactive fragment.

23. The method of any one of claims 18-21, wherein the AK substrate is ATP or AMP.

24. The method of any one of claims 18-21, wherein the AK is selected from the group consisting of AK1, AK2A, AK2B, AK2C, AK3, AK4 and AK5.

25. The method of any one of claims 18-21, wherein the AK is AK3 or AK1.

26. The method of any one of the claims 18-21, wherein the compound affects the interaction of AK with AMP, ATP or AMP and ATP.

27. The method of claim 26, wherein the compound affects the binding of AK with AMP, ATP or AMP and ATP.

28. The method of any one of the claims 18-21, wherein the compound affects the activity of AK on AMP, ATP or AMP and ATP.

29. The method of any one of claims 18-21, wherein the cell overexpresses the AK substrate or the bioactive fragment thereof.

30. The method of any one of claims 18-21, wherein the cell overexpresses AK or the bioactive fragment thereof.

31. The method of any one of claims 18-21, wherein said cell overexpresses the AK substrate or the substrate bioactive fragment and AK or the AK bioactive fragment.

32. The method of any one of the preceding claims, wherein the compound stimulates AMPK activity.

33. The method of any one of the preceding claims, wherein the compound inhibits AMPK activity.

34. A modulator identified by the method of any one of the preceding claims.

35. A method for identifying an AK modulator, comprising contacting a composition comprising AMPK or a bioactive fragment thereof and an AMPK substrate or a bioactive fragment thereof with a test compound and determining the ability of the test compound to affect APMK activity in vitro.

36. A method for identifying an AK modulator, comprising contacting composition comprising AMPK or a bioactive fragment thereof and an AMPK substrate or a bioactive fragment thereof with a test compound and determining the ability of the test compound to affect activity of AMPK activity in the cells.

37. A method for identifying a compound that modulates obesity, comprising contacting a cell or a composition comprising AK or a bioactive fragment thereof and an AK substrate or a bioactive fragment thereof with a test compound and determining the ability of the test compound to modulate an activity of AK or the AK bioactive fragment, to thereby identify an obesity modulator.

38. A method for identifying a compound that modulates obesity, comprising contacting a cell or a composition comprising AMPK or a bioactive fragment thereof and an AMPK substrate or a bioactive fragment thereof with a test compound and determining the ability of the test compound to affect activity of AMPK activity in cells, to thereby identify an obesity modulator.

39. A method for identifying a compound that modulates insulin resistance, comprising contacting a cell or a composition comprising AK or a bioactive fragment thereof and an AK substrate or a bioactive fragment thereof with a test compound and determining the ability of the test compound to modulate an activity of AK or the AK bioactive fragment, to thereby identify an insulin resistance modulator.

40. A method for identifying a compound that modulates insulin resistance, comprising contacting a cell or a composition comprising AMPK or a bioactive fragment thereof and an AMPK substrate or a bioactive fragment thereof with a test compound and determining the ability of the test compound to affect activity of AMPK activity in cells, to thereby identify an insulin resistance modulator

41. The method of any one of claims 37 or 39, wherein the test compound enhances or inhibits energy consumption.

42. The method of any one of claims 35-40, wherein the AK substrate is AMP or ATP.

43. The method of any one of claims 35-40, wherein the AK is selected from the group consisting of AK1, AK2A, AK2B, AK2C, AK3, AK4 and AK5.

44. The method of any one of claims 35-40, wherein the AK is AK3 or AK1.

45. The method of any one of the claims 37 or 39, wherein the compound affects the interaction of AK with AMP, ATP or AMP and ATP.

46. The method of claim 38 or 40, wherein the compound affects the phosphorylation of an AMPK subtrate.

47. The method of any one of the claim 46, wherein the AMPK substrate is AMP, ATP or AMP and ATP.

48. A modulator identified by any one of the claims 45-40.

49. A method of modulating energy impairment in a subject comprising administering to the subject an energy kinase modulator identified according to the methods of any one of claims 1-34 or 35-40 such that energy impairment is modulated.

50. A method of modulating energy consumption in a subject comprising administering to the subject an energy kinase modulator identified according to the methods of any one of claims 1-34 or 35-40 such that energy comsumption is regulated.

51. A method of modulating energy generation in a subject comprising administering to the subject an energy kinase modulator identified according to the methods of any one of claims 1-34 or 35-40 such that energy transmission is regulated.

52. The method of claims 49-51, wherein the subject suffers from type II diabetes or obesity.

53. A pharmaceutical composition comprising the AMPK modulator of claim 34 or 48.

54. A method of treating an energy related disease or disorder comprising administering to a subject the pharmaceutical composition of claim 53.

55. The method of claim 54, wherein the disease or disorder is type II diabetes or obesity.

56. The method of claim 54, wherein the subject is a mammal.

57. The method of claim 54, wherein the subject is human.

58. The method of any one of claims 1-32 wherein the compound stimulates AK activity.

59. The method of any one of claims 1-32 wherein the compound inhibits AK activity.

60. A modulator of AK activity identified by the method of any one of claims 1-32 or 35-36.

61. The method of any one of claims 49-52 and 54-57, wherein the modulator is an inhibitor of AK expression.

62. The method of claim 61, wherein the AK is AK1 or AK3.
